(19) United States
(12) Patent Application Publication
(10) Pub. No.: US 2021/0121666 A1
(43) Pub. Date: Apr. 29, 2021

(54) ILLUMINATING GUIDEWIRE WITH SLIP COUPLING BETWEEN SEGMENTS

(71) Applicant: Acclarent, Inc., Irvine, CA (US)

(72) Inventors: Jetmir Palushi, Irvine, CA (US); Henry F. Salazar, Pico Rivera, CA (US)

(21) Appl. No.: 17/027,773

(22) Filed: Sep. 22, 2020

Related U.S. Application Data
(60) Provisional application No. 62/924,789, filed on Oct. 23, 2019.

(51) Int. Cl. A61M 25/09

(52) U.S. Cl. CPC A61M 25/09; A61M 2205/587; A61M 2025/09091; A61M 2205/02; A61M 2210/065; A61M 2210/0662; A61M 2210/0618

(57) ABSTRACT

A guidewire includes a proximal guidewire portion and a distal guidewire portion. The proximal guidewire portion includes a proximal illumination fiber and a first connection portion. The distal guidewire portion includes at least one distal illumination fiber and a second connection portion. The first connection portion is configured to couple to the second connection portion to define a rotatable slip coupling. The distal guidewire portion is configured to rotate about the longitudinal axis relative to the proximal guidewire portion at the slip coupling. The slip coupling provides optical continuity between the proximal illumination fiber and the at least one distal illumination fiber. The slip coupling may include a rotation limiting feature configured to limit the rotation of the distal guidewire portion about the longitudinal axis relative to the proximal guidewire portion.

### PRIORITY

This application claims priority to U.S. Provisional Pat. App. No. 62/924,789, entitled “Illuminating Guidewire with Slip Coupling Between Segments,” filed Oct. 23, 2019, the disclosure of which is incorporated by reference herein.

### BACKGROUND

In some instances, it may be desirable to dilate an anatomical passageway in a patient. This may include dilation of ostia of paranasal sinuses (e.g., to treat sinusitis), dilation of the larynx, dilation of the Eustachian tube, dilation of other passageways within the ear, nose, or throat, etc. One method of dilating anatomical passageways includes using a guide wire and catheter to position an inflatable balloon within the anatomical passageway, then inflating the balloon with a fluid (e.g., saline) to dilate the anatomical passageway. For instance, the expandable balloon may be positioned within an ostium at a paranasal sinus and then be inflated, to thereby dilate the ostium by remodeling the bone adjacent to the ostium, without requiring incision of the mucosa or removal of any bone. The dilated ostium may then allow for improved drainage from and ventilation of the affected paranasal sinus. A system that may be used to perform such procedures may be provided in accordance with the teachings of U.S. Pub. No. 2011/0004057, entitled “Systems and Methods for Transnasal Dilation of Passageways in the Ear, Nose or Throat,” published Jan. 6, 2011, now abandoned, the disclosure of which is incorporated by reference herein. An example of such a system is the Relieva® Spin Balloon Sinuplasty System by Acclarent, Inc. of Irvine, Calif.

In the context of Eustachian tube dilation, a dilation catheter or other dilation instrument may be inserted into the Eustachian tube and then be inflated or otherwise expanded to thereby dilate the Eustachian tube. The dilated Eustachian tube may provide improved ventilation from the nasopharynx to the middle ear and further provide improved drainage from the middle ear to the nasopharynx. Methods and devices for dilating the Eustachian tube are disclosed in U.S. Pat. Pub. No. 2010/0274188, entitled “Method and System for Treating Target Tissue within the ET,” published on Oct. 28, 2010, now abandoned, the disclosure of which is incorporated by reference herein; and U.S. Pat. Pub. No. 2013/0274715, entitled “Method and System for Eustachian Tube Dilation,” published on Oct. 17, 2013, now abandoned, the disclosure of which is incorporated by reference herein. An example of such a system is the Aera® Eustachian Tube Balloon Dilation System by Acclarent, Inc. of Irvine, Calif.

While a variable direction view endoscope may be used to provide visualization within the anatomical passageway, it may also be desirable to provide additional visual confirmation of the proper positioning of the balloon before inflating the balloon. This may be done using an illuminating guidewire. Such a guidewire may be positioned within the target area and then illuminated, with light projecting from the distal end of the guidewire. This light may illuminate the adjacent tissue (e.g., hypodermis, subdermis, etc.) and thus be visible to the naked eye from outside the patient through transcutaneous illumination. For instance, when the distal end is positioned in the maxillary sinus, the light may be visible through the patient's cheek. Using such external visualization to confirm the position of the guidewire, the balloon may then be advanced distally along the guidewire into position at the dilation site. Such an illuminating guidewire may be provided in accordance with the teachings of U.S. Pat. No. 9,155,492, entitled “Sinus Illumination Lightwire Device,” issued Oct. 13, 2015, the disclosure of which is incorporated by reference herein. An example of such an illuminating guidewire is the Relieva Luma Sentry® Sinus Illumination System by Acclarent, Inc. of Irvine, Calif.

Image-guided surgery (IGS) is a technique where a computer is used to obtain a real-time correlation of the location of an instrument that has been inserted into a patient's body to a set of preoperatively obtained images (e.g., a CT or MRI scan, 3-D map, etc.), such that the computer system may superimpose the current location of the instrument on the preoperatively obtained images. An example of an electromagnetic IGS navigation systems that may be used in IGS procedures is the CARTO® 3 System by Biosense-Webster, Inc., of Irvine, Calif. In some IGS procedures, a digital tomographic scan (e.g., CT or MRI, 3-D map, etc.) of the operative field is obtained prior to surgery. A specially programmed computer is then used to convert the digital tomographic scan data into a digital map. During surgery, special instruments having sensors (e.g., electromagnetic coils that emit electromagnetic fields and/or are responsive to externally generated electromagnetic fields) are used to perform the procedure while the sensors send data to the computer indicating the current position of each surgical instrument. The computer correlates the data it receives from the sensors with the digital map that was created from the preoperative tomographic scan. The tomographic scan images are displayed on a video monitor along with an indicator (e.g., crosshairs or an illuminated dot, etc.) showing the real-time position of each surgical instrument relative to the anatomical structures shown in the scan images. The surgeon is thus able to know the precise position of each sensor-equipped instrument by viewing the video monitor even if the surgeon is unable to directly visualize the instrument itself at its current location within the body.

An example of an electromagnetic IGS systems that may be used in ENT and sinus surgery is the CARTO® 3 System by Biosense-Webster, Inc., of Irvine, Calif. When applied to functional endoscopic sinus surgery (FESS), balloon sinuplasty, and/or other ENT procedures, the use of IGS systems allows the surgeon to achieve more precise movement and positioning of the surgical instruments than can be achieved by viewing through an endoscope alone. As a result, IGS systems may be particularly useful during performance of FESS, balloon sinuplasty, and/or other ENT procedures where anatomical landmarks are not present or are difficult to visualize endoscopically. In order to enable use of an IGS system in an ENT procedure, the instrumentation used in the ENT procedure may include a guidewire that has a position sensor that cooperates with the IGS system to provide data indicating the position of the distal end of the guidewire in real time. Such a IGS system navigation guidewire may be used in addition to, or in lieu of, the navigating guidewire referred to above. Examples of use of an IGS system in an ENT procedure are described in U.S. Pat. Pub. No. 2014/0364725, entitled “Systems and Methods for Performing Image Guided Procedures within the Ear, Nose, Throat and Paranasal Sinuses,” published Dec. 11, 2014, now abandoned, the disclosure of which is incorporated by reference herein; and U.S. Pat. No. 10,561,370, entitled “Apparatus to Secure Field Generating Device to Chair,” issued Feb. 18, 2020, the disclosure of which is incorporated by reference herein.

While several systems and methods have been made and used to position a balloon of a dilation catheter in an anatomical passageway, it is believed that no one prior to the inventors has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1A depicts a perspective view of an exemplary dilation instrument, with a guidewire and a dilation catheter of the instrument each in respective proximal positions;

FIG. 1B depicts a perspective view of the dilation instrument of FIG. 1A, with the guidewire in a distal position and the dilation catheter in the proximal position;

FIG. 1C depicts a perspective view of the dilation instrument of FIG. 1A, with the guidewire and the dilation catheter each in respective distal positions, and with a dilator of the dilation catheter in a non-expanded state;

FIG. 1D depicts a perspective view of the dilation instrument of FIG. 1A, with the guidewire and the dilation catheter each in respective distal positions, and with a dilator of the dilation catheter in an expanded state;

FIG. 2 depicts a perspective view of a guidewire actuation assembly of the dilation instrument of FIG. 1A;

FIG. 3 depicts an enlarged perspective view of actuators of the guidewire actuation assembly of FIG. 2, with a collet collar in a proximal position;

FIG. 4 depicts an enlarged perspective view of actuators of the guidewire actuation assembly of FIG. 2, with the collet collar of FIG. 3 in a distal position;

FIG. 5 depicts a perspective view of a spin actuator of the guidewire actuation assembly of FIG. 2;

FIG. 6 depicts a side elevation view of an exemplary illumination guidewire of the dilation instrument of FIG. 1A, with the proximal and distal portions of the illumination guidewire joined by a slip coupling;

FIG. 7 depicts an enlarged perspective view of the slip coupling of the illumination guidewire of FIG. 6, with the proximal and distal portions of the illumination guidewire separated and the proximal portion shown as transparent for greater clarity;

FIG. 8 depicts an enlarged perspective view of the slip coupling of the illumination guidewire of FIG. 6, with the proximal and distal portions of the illumination guidewire separated and the distal portion shown as transparent for greater clarity; and

FIG. 9 depicts an enlarged perspective view of a slip coupling of another exemplary illumination guidewire, with the proximal and distal portions of the illumination guidewire separated and the proximal portion shown as transparent for greater clarity.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. For example, while various. Accordingly, the drawings and descriptions should be regarded as illustrative in nature and not restrictive.

It will be appreciated that the terms “proximal” and “distal” are used herein with reference to a clinician gripping a handpiece assembly. Thus, an end effector is distal with respect to the more proximal handpiece assembly. It will be further appreciated that, for convenience and clarity, spatial terms such as “circumferentially” and “radially” also are used herein with respect to the longitudinal axis. However, surgical instruments are used in many orientations and positions, and these terms are not intended to be limiting and absolute.

It is further understood that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

I. Exemplary Dilation Instrument

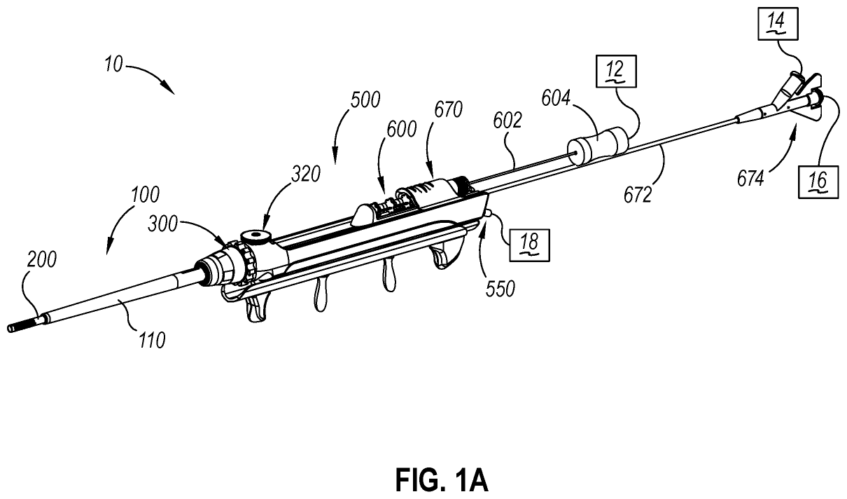

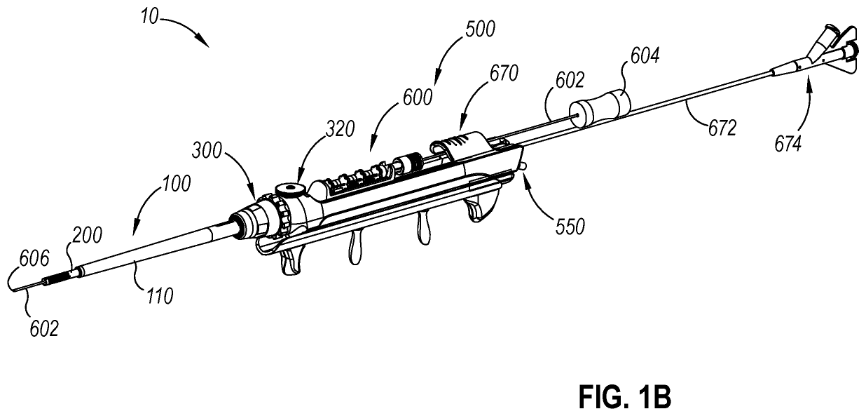

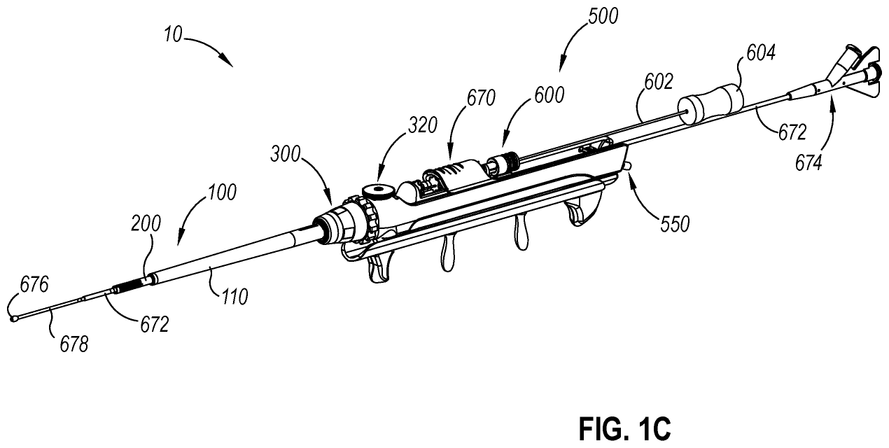

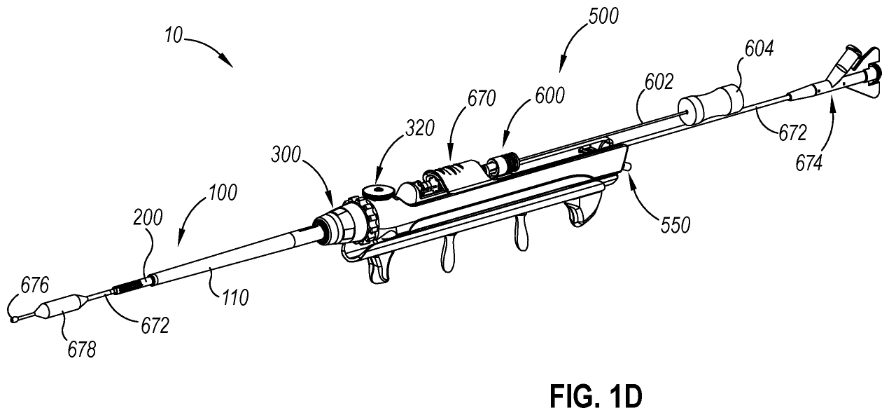

FIGS. 1A-1D show an exemplary dilation instrument (10) that may be used to dilate the ostium of a paranasal sinus, to dilate another passageway associated with drainage of a paranasal sinus, to dilate a Eustachian tube, or to dilate some other anatomical passageway (e.g., within the ear, nose, or throat, etc.). Dilation instrument (10) of the present example provides adjustability that enables the operator to use dilation instrument (10) in different scenarios, without requiring the operator to switch between different instruments. For instance, dilation instrument (10) may be used to dilate various different anatomical passageways (e.g., frontal sinus ostium, frontal recess, maxillary sinus ostium, sphenoid sinus ostium, ethmoid sinus ostium, Eustachian tube, etc.) by making simple adjustments to structural features of the instrument.

A. Overview of Dilation Instrument

Dilation instrument (10) of this example includes a handle assembly (500), a guide shaft assembly (100) extending distally from handle assembly (500); a guidewire actuation assembly (600) slidably coupled with handle assembly (500); and a dilation catheter actuation assembly (670) slidably coupled with handle assembly (500). A guidewire module (12) is coupled with a distal portion of a guidewire (602) of dilation instrument (10) via a connector (604). An inflation fluid source (14) and an irrigation fluid source (16) are coupled with a dilation catheter (672) of dilation instrument (10) via a connector (674). A suction source (18) is coupled with a suction conduit (802) (FIG. 14) of dilation instrument (10) via a suction port (550).

Handle assembly (500) is sized and configured to be grasped and operated by a single hand of an operator. The operator may selectively operate guidewire actuation assembly (600) and dilation catheter actuation assembly (670) with the same single hand that grasps handle assembly (500). As shown in the transition from FIG. 1A to FIG. 1B, the operator may advance guidewire actuation assembly (600) distally along handle assembly (500) to thereby advance guidewire (602) distally, such that distal end (606) of guidewire (602) is positioned distal to distal end of guide shaft assembly (100). As shown in the transition from FIG. 1B to FIG. 1C, the operator may advance dilation catheter actuation assembly (670) distally along handle assembly (500) to thereby advance dilation catheter (672) distally, such that distal tip (676) of dilation catheter (672) is positioned distal to distal end of guide shaft assembly (100). With dilation catheter (672) advanced to a distal position, the operator may then inflate a dilator (678) of dilation catheter (672) to achieve an expanded state as shown in FIG. 1D, to thereby dilate an anatomical passageway in which dilator (678) is positioned.

Guide shaft assembly (100) of this example includes a rigid shaft member (110), a flexible shaft member (200), and a deflection control knob (300). Deflection control knob (300) is operable to cause guide shaft assembly (100) to flex laterally at flexible guide shaft member (200), to thereby allow the operator to vary the exit angle of dilation catheter (672) relative to the longitudinal axis of rigid shaft member (110). A rotation control knob (320) is operable to rotate guide shaft assembly (100) about the longitudinal axis of rigid shaft member (110), thereby providing additional control to the operator to facilitate access to various anatomical passageways within the head of a patient.

In the present example, dilation catheter (672) is coaxially disposed within guide shaft assembly (100), and guidewire (602) is coaxially disposed within dilation catheter (672). In some other versions, guide shaft assembly (100) is coaxially disposed within dilation catheter (672), and guidewire (602) is coaxially disposed within guide shaft assembly (100). Also, in some versions, guidewire (602) is omitted.

The proximal end of dilation catheter (672) includes a connector (674) that is configured to couple with an inflation fluid source (14) and an irrigation fluid source (16). By way of example only, connector (674) may be connected and operable in accordance with at least some of the teachings of U.S. Pat. Pub. No. 2019/0274701, entitled “Fluid Fitting for Dilation Instrument,” published Sep. 12, 2019, the disclosure of which is incorporated by reference herein. Inflation fluid source (14) is operable to provide an inflation fluid (e.g., saline) via connector (674) to selectively inflate and deflate dilator (678) of dilation catheter (672). In some versions, inflation fluid source (14) is constructed and operable in accordance with at least some of the teachings of U.S. Pat. No. 9,962,530, entitled “Inflator for Dilation of Anatomical Passageway,” issued May 8, 2018, the disclosure of which is incorporated by reference herein. As another merely illustrative example, inflation fluid source (14) may be constructed and operable in accordance with at least some of the teachings of U.S. Pub. No. 2016/0058985, entitled “Automated Inflator for Balloon Dilator,” published Mar. 3, 2016, now abandoned, the disclosure of which is incorporated by reference herein. Other suitable forms that inflation fluid source (14) may take will be apparent to those skilled in the art in view of the teachings herein.

In addition to being capable of providing dilation, dilation catheter (672) of the present example is also configured to provide irrigation of a site within a patient. By way of example only, dilation catheter (672) may be constructed and operable in accordance with at least some of the teachings of U.S. Pat. No. 9,095,646, entitled “Devices and Methods for Transnasal Dilation and Irrigation of the Sinuses,” issued Aug. 4, 2015, the disclosure of which is incorporated by reference herein. Dilation catheter (672) receives irrigation fluid (e.g., saline) from irrigation fluid source (16) via connector (674) of connector (674) as described above. By way of example only, irrigation fluid source (16) may provide gravity-fed irrigation fluid, may include a syringe, may include an electrically activated pump, or may take any other suitable form as will be apparent to those skilled in the art in view of the teachings herein.

By way of further example only, dilation instrument (10) may be further configured and operable in accordance with the teachings of U.S. Pat. Pub. No. 2019/0015645, entitled “Adjustable Instrument for Dilation of Anatomical Passageway,” published Jan. 17, 2019, the disclosure of which is incorporated by reference herein; or in accordance with the teachings of any other patent reference cited herein. Other variations of the features and functionalities described herein will be apparent to those skilled in the art in view of the teachings herein.

B. Exemplary Guidewire and Associated Actuation Assembly of Dilation Instrument

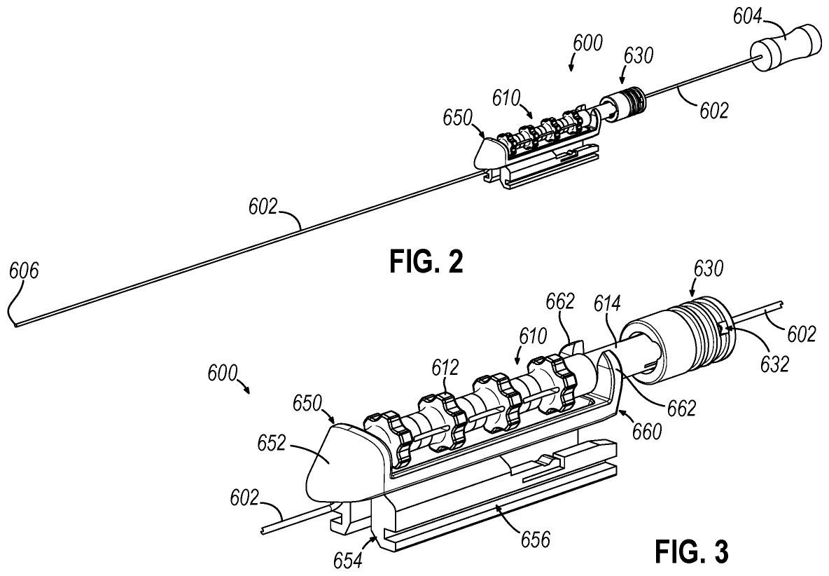

FIGS. 2-5 show various components of guidewire actuation assembly (600) in greater detail. These components include a spin actuator (610) and a slide actuator (650). Spin actuator (610) is operable to rotate guidewire (602) relative to handle assembly (500), about longitudinal axis of guidewire (602); while slide actuator (650) is operable to translate guidewire (602) relative to handle assembly (500), along the longitudinal axis of guidewire (602).

In some versions, guidewire (602) includes an outer coil and includes one or more internal optical fibers (not shown) and a distal end (606) that is configured to emit visible light. In some such versions, guidewire module (12) includes a light source, and connector (604) is operable to communicate light from the light source of guidewire module (12) to guidewire (602). Illuminating versions of guidewire (602) may be used to provide position confirmation through observation of transillumination effects. By way of example only, illuminating versions of guidewire (602) may be constructed and operable in accordance with at least some of the teachings of U.S. Pat. No. 9,155,492, entitled “Sinus Illumination Lightwire Device,” issued Oct. 13, 2015, the disclosure of which is incorporated by reference herein.

In addition to providing illumination, or as an alternative to providing illumination, guidewire (602) may provide position sensing capabilities. In some such versions, the distal end of guidewire (602) may include a position sensor. By way of example only, such a guidewire (602) may be constructed and operable in accordance with at least some of the teachings of U.S. Pat. No. 10,610,308, entitled “Navigation Guidewire with Interlocked Coils,” issued Apr. 7, 2020, the disclosure of which is incorporated by reference herein; U.S. Pat. Pub. No. 2019/0192177, entitled “Reusable Navigation Guidewire,” published Jun. 27, 2019, the disclosure of which is incorporated by reference herein; U.S. Pat. No. 10,463,242, entitled “Guidewire Navigation for Sinuplasty,” issued Nov. 5, 2019, the disclosure of which is incorporated by reference herein. In some such versions, guidewire module (12) includes an IGS navigation system, and connector (604) is operable to communicate position-indicative signals from the sensor of guidewire (602) to guidewire module (12).

As best seen in FIG. 5, spin actuator (610) of the present example includes a plurality of thumbwheel engagement features (612), a proximal shaft (614), and a distal shaft (616). Proximal shaft (614) includes a collet chuck feature (640) formed by a pair of collet leaves (620) that are separated by diametrically opposed longitudinally extending slots (622). Slots (622) are configured to provide clearance to allow collet leaves (620) to deflect inwardly toward each other to thereby grip guidewire (602). Each collet leaf (620) includes a fin (624) extending longitudinally and radially outwardly. Each fin (624) includes a proximally positioned detent feature (626).

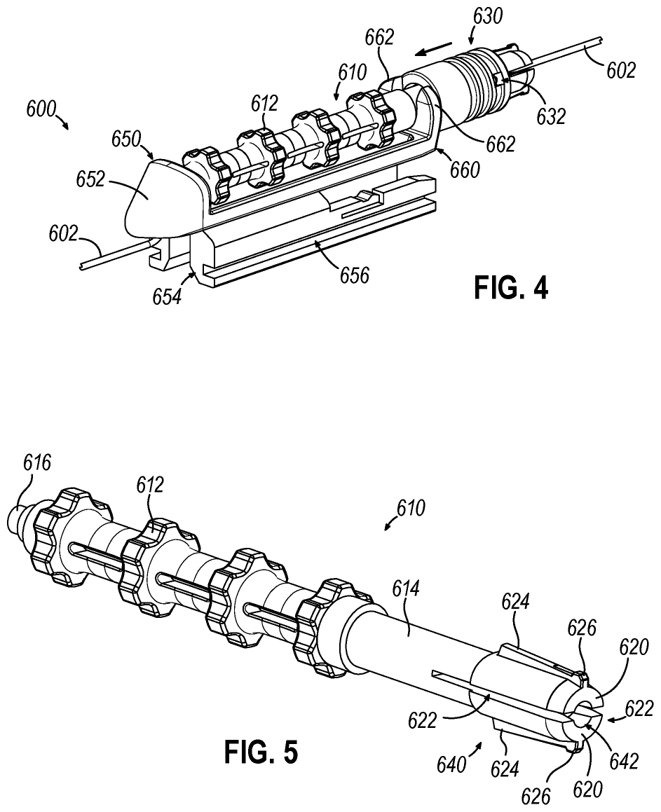

As shown in FIGS. 3-4, a collet collar (630) is configured to translate along proximal shaft (614) between a proximal position (FIG. 3) and a distal position (FIG. 4) to thereby transition collet chuck feature (640) between a locked state (FIG. 3) and an unlocked state (FIG. 4). The interior of collet collar (630) includes features that are operable to provide camming to drive leaves (620) inwardly toward each other as collet collar (630) is translated from the distal position to the proximal position. Detent features (626) of collet chuck feature (640) cooperate with notches (632) of collet collar (630) to selectively maintain the longitudinal position of collet collar (630) along proximal shaft (614) when collet collar (630) is in the proximal position. Detent features (626) thus cooperate with notches (632) to selectively maintain collet chuck feature (640) in the locked state.

When collet chuck feature (640) is in the locked state, collet leaves (620) are deformed inwardly to grip guidewire (602) disposed in the central longitudinal bore (642) of spin actuator (610). When collet chuck feature (640) is in the unlocked state, collet leaves (620) resiliently return to their natural position, thereby releasing their grip on guidewire (602). Thus, when collet chuck feature (640) is in the unlocked state, the operator may selectively adjust the longitudinal position of guidewire (602) relative to spin actuator (610). In some instances, the operator may wish to remove guidewire (602) from spin actuator (610) when collet chuck feature (640) is in the unlocked state. In some such instances, the operator may wish to exchange one guidewire (602) for another guidewire (602) (e.g., to exchange an illuminating guidewire (602) for a guidewire (602) having a position sensor, or vice-versa, etc.).

As best seen in FIGS. 3-4, slide actuator (650) of the present example comprises a distal nose portion (652), a lower base portion (654), and a proximal yoke (660). Distal nose portion (652) is configured to rotatably support distal shaft (615) of spin actuator (610). Proximal yoke (660) includes a pair of fork tines (662) that are configured to rotatably support proximal shaft (614) of spin actuator (610). Lower base portion (654) includes a pair of longitudinally extending recesses (656) that are configured to slidably receive corresponding rails (not shown) defined by housings (540) of handle assembly (500). Slide actuator (650) is operable to slide longitudinally relative to housings (540), to thereby translate guidewire (602) and spin actuator (610) longitudinally, while also allowing spin actuator (610) to rotate guidewire (602) relative to slide actuator (650). Distal nose portion (652) is also configured to redirect guidewire (602) from a first longitudinal axis (associated with the proximal portion of guidewire (602)) to a second longitudinal axis (associated with dilation catheter (672) and the distal portion of guidewire (602), with second longitudinal axis being parallel with the first longitudinal axis.

As noted above with reference to FIGS. 1A-1B, an operator may translate guidewire (602) longitudinally relative to handle assembly (500) by engaging guidewire actuation assembly (600) and sliding guidewire actuation assembly (600) longitudinally along handle assembly (500). Due to the position and configuration of guidewire actuation assembly (600), the operator may accomplish such motion by simply engaging guidewire actuation assembly (600) with the thumb (or another finger) of the hand that is grasping handle assembly (500). In some instances, the operator may also wish to rotate guidewire (602) about longitudinal axis of guidewire (602). This may be particularly desirable when distal end of guidewire (602) includes a preformed bend, as rotation of guidewire (602) may be used to advantageously reorient the bent distal end of guidewire (602) to thereby align the bent distal end of guidewire (602) with a targeted passageway. To provide such rotation, the operator may engage one or more thumbwheel engagement features (612) with the thumb (or another finger) of the hand that is grasping handle assembly (500). Guidewire actuation assembly (600) is thus configured to facilitate single-handed use including translation and rotation of guidewire (602). The elongate configuration of guidewire actuation assembly (600) may further facilitate single-handed use regardless of whether guidewire actuation assembly (600) is positioned distally or proximally along handle assembly (500).

II. Exemplary Guidewire with Threaded Slip Coupling Between Segments

In some instances, it may be desirable to provide a version of guidewire (602) that includes features that enhance the rotational functionality of guidewire (602) such that guidewire (602) may rotate about its longitudinal axis without damaging any components housed within guidewire (602). For example, in some versions, one or more core wires and optical fibers traverse an inner lumen of guidewire (602). Such core wires may be non-extensible, such that a core wire may be configured to prevent stretching of guidewire (602) that might otherwise occur in the absence of such a core wire. As guidewire (602) is rotated about its longitudinal axis, the core wire(s) and/or optical fiber(s) can in some instances become twisted, potentially damaging either internal component. In addition, or in the alternative, rotation of guidewire (602) about the longitudinal axis of guidewire (602) may result in winding, or a build-up of torsion, along at least a portion of the length of guidewire (602). Such winding or build-up of torsion may ultimately result in an unpredictable whipping or spring-back action in guidewire (602) as the torsion is eventually released. It may therefore be desirable to provide a version of guidewire (602) where only a distal segment of the guidewire is rotated while a proximal segment of the guidewire remains stationary; while still maintaining optical continuity along the entire length of the guidewire so that transillumination light may be communicated form the proximal end of the guidewire to the distal end of the guidewire.

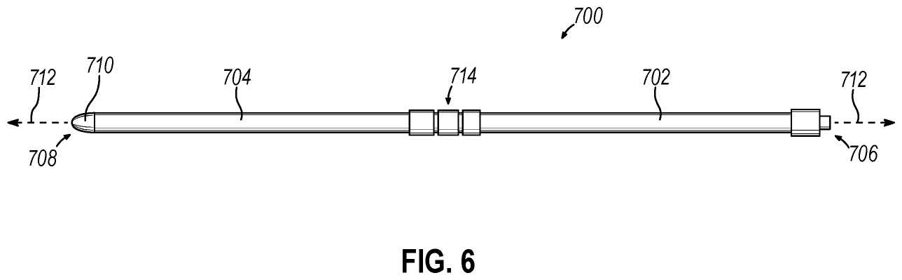

FIG. 6 shows another exemplary guidewire (700) that may be incorporated into instrument (10). Guidewire (700), or more specifically the distal portion (704) of guidewire (700), may be included in place guidewire (602) as shown in FIGS. 1A-4 and may provide substantially the same or similar functionality, except as otherwise described below. As shown, guidewire (700) of this example includes a proximal portion (702) and a distal portion (704) configured to coaxially align and slidably couple together, thereby defining a common longitudinal axis (712). Proximal portion (702) includes an outer sheath (730) with a hollow core that defines a lumen (734). Distal portion (704) also includes an outer sheath (720) with a hollow core that defines a lumen (724). By way of example only, outer sheath (720) and/or outer sheath (730) may include helical coil formed of metallic or polymeric wire. Alternatively, outer sheath (720) and/or outer sheath (730) may include a metallic or polymeric mesh structure. As another merely illustrative alternative, outer sheath (720) and/or outer sheath (730) may include a cylindrical body. Other suitable forms that each outer sheath (720, 730) may take will be apparent to those skilled in the art in view of the teachings herein.

Proximal end (706) of proximal portion (702) may couple to optical light source, and distal end (708) of distal portion (704) may permit light from the optical light source to emanate in one or more directions outwardly from distal end (708). Further, distal end (708) of distal portion (704) can include an atraumatic lens (710) to distribute or disperse the light provided by the light source. As will be described in greater detail below, the one or more optical fibers may be disposed within lumens (724, 734) of proximal portion (702) and distal portion (704) of guidewire (700) for carrying the light from the light source to the atraumatic lens (710).

In the present example, distal portion (704) is configured to rotate about longitudinal axis (712) relative to proximal portion (702), such as by a user actuating spin actuator (610). A connector (714), which may take the place of connector (604) as described above, enables optical and rotatable coupling between proximal portion (702) and distal portion (704). Connector (714) maintains optical continuity between proximal portion (702) and distal portion (704) while permitting rotation of distal portion (704) relative to proximal portion (702). In some versions, distal portion (704) of guidewire (700) is more flexible than proximal portion (702) of guidewire (700). Distal portion (704) may be gripped by spin actuator (610) such that rotation of spin actuator (610) relative to slide actuator (650) (and other components of handle assembly (500)) will cause rotation of distal portion (704) relative to slide actuator (650) (and other components of handle assembly (500)). Proximal portion (702) may remain stationary relative to slide actuator (650) (and other components of handle assembly (500)) while distal portion (704) rotates relative to slide actuator (650) (and other components of handle assembly (500)).

Guidewire (700) has a length enabling distal end (708) of distal portion (704) of guidewire (700) to be positioned distal to dilator (678) while proximal end (706) of proximal portion (702) of guidewire (700) is positioned proximal to handle assembly (500). Guidewire (700) may include indicia along at least part of its length to provide the operator with visual feedback indicating the depth of insertion of guidewire (700) relative to dilation catheter (672). By way of example only, at least a portion of guidewire (700) may be configured and operable in accordance with at least some of the teachings of U.S. Pat. No. 9,155,492, entitled “Sinus Illumination Lightwire Device,” issued October 13, 2015, the disclosure of which is incorporated by reference herein. In some versions, at least a portion of guidewire (700) is configured and operable similar to the Relieva Luma Sentry® Sinus Illumination System by Acclarent, Inc. of Irvine, Calif. Other suitable forms that guidewire (700) may take will be apparent to those of ordinary skill in the art in view of the teachings herein.

With reference to FIGS. 6-8, connector (714) of some versions of guidewire (700) may be in the form of a slip coupling. Such a slip coupling may be configured to provide tensile strain relief for guidewire (700) by allowing distal portion (704) of guidewire (700) to freely rotate about longitudinal axis (712) relative to proximal portion (702) via connector (714). This may prevent the build-up of torsion along any components of guidewire housed within proximal portion (702) of guidewire (700) while distal portion (704) of guidewire (700) is rotated about the longitudinal axis (712). For example, in versions where guidewire (700) includes one or more optical fibers or other light-communicating features, connector (714) includes features allowing light to pass freely through connector (714), such that connector (714) maintains electrical and/or optical continuity between guidewire module (12) and optical fibers spanning through both the proximal portion (702) and distal portion (704) of guidewire (700).

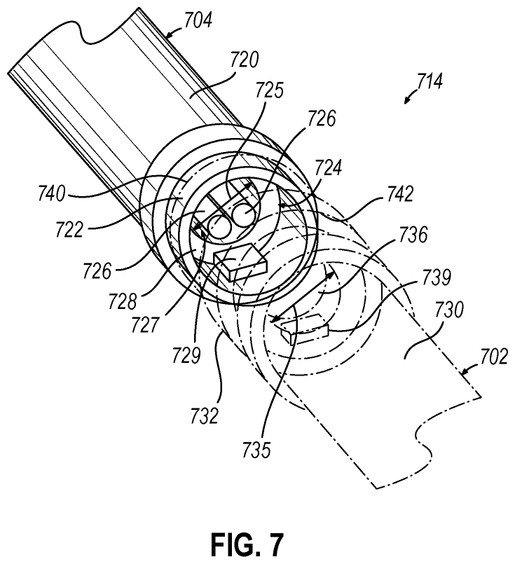

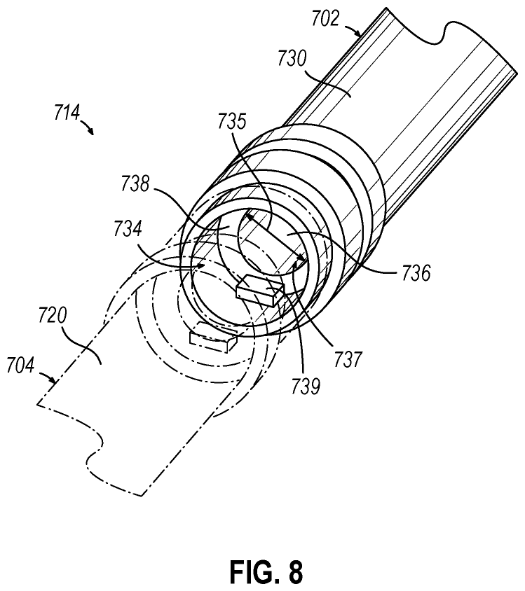

FIGS. 7-8 show connector (714) of the present example in greater detail. Connector (714) may be formed by a pair of mating halves of proximal portion (702) and distal portion (704) or any other suitable components. In some versions, distal portion (704) can include a flange (722) configured to fit into and slidably mate with a slip fitting (732) of proximal portion (702). In other versions, proximal portion (702) and distal portion (704) can include relatively flat surfaces configured to be surrounded by a slip covering. By way of further example only, components of connector (714) that rotatably secure proximal portion (702) to distal portion (704) will be apparent to those of ordinary skill in the art in view of the teachings herein. Components of connector (714) may be formed of metal, plastic, composite, ceramic, and/or various other kinds of materials.

With reference to FIG.7, lumen (724) of distal portion (704) includes one or more optical fibers (726) for transmitting light from the light source to the atraumatic lens (710). Lumen (724) may include any number of optical fibers (726). In some versions, multiple optical fibers (726), defining an optical cable, may be disposed within lumen (724) to permit distal portion (704) to remain laterally flexible. Proximal end (727) of optical fibers (726), whether a single fiber or a bundle of fibers, define a fiber diameter (725) within lumen (724). In addition to optical fibers (726), lumen (724) may include one or more core wires (728), and core wire (728) may be coupled to the inner surface of distal portion (704) via a joint (729), such as a solder joint or any other suitable adhesive, to provide adequate structural integrity to distal portion (704).

With reference to FIG.8, lumen (734) of proximal portion (702) includes one or more optical fibers (736) for carrying light from the light source to the atraumatic lens (710) of distal portion (704). Lumen (734) may include any number of optical fibers (736). In some versions, multiple optical fibers (736), defining an optical cable, may be disposed within lumen (734) to permit proximal portion (702) to remain laterally flexible, while in other versions, a single optical fiber (736) may be disposed within lumen (734), as shown. Distal end (737) of optical fibers (736), whether a single fiber or a bundle of fibers, define a fiber diameter (735) within lumen (734). In addition to optical fiber (736), lumen (734) may include one or more core wires (738), and core wire (738) may be coupled to the inner surface of proximal portion (702) via a joint (739), such as a solder joint or any other suitable adhesive, to provide adequate structural integrity to proximal portion (702).

Upon connecting proximal portion (702) to distal portion (704) of guidewires together via connector (714), wherein connector (714) takes the form of a slip coupling, proximal ends (727) of optical fibers (726) of distal portion (704) are in optical communication with distal end (737) of optical fiber (736) of proximal portion (702). To maximize the light that passes from proximal portion (702) to distal portion (704), fiber diameter (725) of distal portion (704) can be configured to be equal to or lesser than fiber diameter (735) of proximal portion (702), such that optical fibers (726, 736) are positioned in a concentric arrangement. As such, as distal portion (704) is rotated about longitudinal axis (712) relative to proximal portion (702), a maximum amount of light passes between proximal portion (702) and distal portion (704) without requiring one continuous optical fiber running from proximal portion (702) through to distal portion (704), potentially resulting in the one continuous optical fiber and/or one continuous core wire creating torsional tension within guidewire (700). In this configuration, distal portion (704) may rotate freely about longitudinal axis (712) relative to proximal portion (702).

In some versions, connector (714) can include a rotation limiting feature to ensure distal portion (704) is only permitted to rotate freely about longitudinal axis (712) relative to proximal portion (702) for a predefined number of revolutions about longitudinal axis (712). In some versions, rotation limiting feature can include a threaded connection, such that slip fitting (732) of proximal portion (702) can include a threading (742) configured to engage with a threading (740) on flange (722) of distal portion (704). Such a rotation limiting feature can, for example, provide a rotational limit of distal portion (704) relative to proximal portion (702) of between two and four revolutions in the same rotational direction. In other examples, a rotation limiting feature can provide a rotational limit of distal portion (704) relative to proximal portion (702) of less than two revolutions or more than four revolutions, as necessary.

In the present example, proximal portion (702) includes just one single optical fiber (736) while distal portion (704) includes two optical fibers (726). In some other versions, distal portion (704) includes more than two optical fibers (726). By including two or more relatively small diameter optical fibers (726) instead of one single relatively large diameter optical fiber, distal portion (704) may provide more room for other components (e.g., core wire (728), a wire to a position sensor (not shown), etc.) in distal portion (704). Including two or more relatively small diameter optical fibers (726) in distal portion (704) may also provide better bendability, including increased flexibility and reduced risk of kinking, as compared to a version of distal portion (704) having one single relatively large diameter optical fiber.

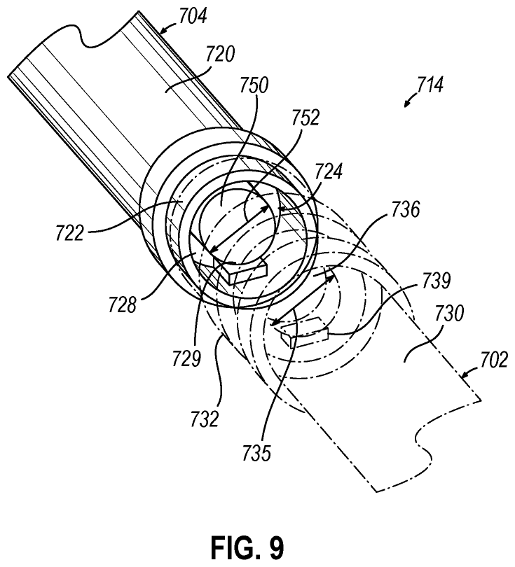

FIG. 9 shows guidewire (700) with a single optical fiber (750) disposed within lumen (724) of distal portion (704). Single optical fiber (750) can be included in place of the multiple optical fibers (726) shown in FIG. 7. Similar to optical fibers (726), optical fiber (750) defines an optical diameter (752) that is equal to or less than optical diameter (735) of optical fiber (736) of proximal portion (702). Further, optical fibers (750, 736) are coaxially aligned and positioned in a concentric arrangement. Therefore, as distal portion (704) is rotated about longitudinal axis (712) relative to proximal portion (702), a maximum amount of light passes between proximal portion (702) and distal portion (704) without requiring one continuous optical fiber running from proximal portion (702) through to distal portion (704), potentially resulting in the one continuous optical fiber and/or one continuous core wire creating torsional tension within guidewire (700). As should be understood, optical fibers (726, 736, 750) can be configured with any suitable number of optical fibers, or optical cables defined by multiple optical fibers, where each optical fiber defines an optical diameter supporting the concentric arrangement between the optical fiber (736) of proximal portion (702) and optical fiber (726, 750) of distal portion (704) to permit the maximum amount of light to travel from light source to atraumatic lens (710) as distal portion (704) is rotated.

Some versions of guidewire (700) may include a position sensor (not shown) in distal end (708) of distal portion (704). Such a position sensor may be configured to generate signals in response to an alternating magnetic field, with such signals indicating the position of distal end (708) of distal portion (704) in three-dimensional space as is known in the art. In such versions, connector (714) may also include an electrical slip coupling that provides electrical continuity between a signal carrying wire, trace, or other conductor(s) of distal portion (704) and a corresponding wire, trace, or other conductor(s) of proximal portion (702). Thus, connector (714) may enable signals from a position sensor to be communicated from distal portion (704) to an IGS navigation system that is coupled with proximal portion (702), in addition to providing the optical continuity between portions (702, 704) as described above; and in addition to permitting distal portion (702) to rotate relative to proximal portion (704).

While various examples described herein are provided in the context of instrumentation that is sized to pass through a paranasal sinus ostium, it should be understood that this is just a merely illustrative example. The teachings herein may be readily applied in the context of instrumentation that is positioned anywhere within a patient's head; or elsewhere within a patient's anatomy. Other examples of anatomical structures that may be reached by instrumentation configured in accordance with the teachings herein include, but are not limited to, cranial nerves, vidian nerves, etc. Still other examples of anatomical structures that may be reached by instrumentation configured in accordance with the teachings herein will be apparent to those skilled in the art in view of the teachings herein.

III. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### EXAMPLE 1

A guidewire, comprising: (a) a proximal guidewire portion, including: (i) a first lumen, (ii) a proximal illumination fiber disposed within the first lumen, and (iii) a first connection portion at a distal end of the proximal guidewire portion; and (b) a distal guidewire portion configured to coaxially align with the proximal guidewire portion to define a longitudinal axis, including: (i) a second lumen, (ii) at least one distal illumination fiber disposed within the second lumen, and (iii) a second connection portion at a proximal end of the distal guidewire portion; wherein the first connection portion is configured to couple to the second connection portion to define a rotatable slip coupling; wherein the distal guidewire portion is configured to rotate about the longitudinal axis relative to the proximal guidewire portion at the slip coupling; wherein the slip coupling is configured to provide optical continuity between the proximal illumination fiber and the at least one distal illumination fiber; and wherein the slip coupling includes a rotation limiting feature configured to limit the rotation of the distal guidewire portion about the longitudinal axis relative to the proximal guidewire portion.

### EXAMPLE 2

The guidewire of Example 1, further comprising an illumination source coupled to a proximal end of the proximal illumination fiber.

### EXAMPLE 3

The guidewire of Example 2, wherein a distal end of the distal guidewire portion includes an atraumatic lens configured to emanate light from the illumination source.

### EXAMPLE 4

The guidewire of any of Examples 1-3, wherein the slip coupling is configured to provide electrical continuity between the proximal guidewire portion and the guidewire portion.

### EXAMPLE 5

The guidewire of any of Examples 1-4, further comprising: (a) a first threading defined by the first connection portion; and (b) a second threading defined by the second connection portion and configured to form a threaded connection with the first threading.

### EXAMPLE 6

The guidewire of Example 5, wherein the rotation limiting feature includes the threaded connection.

### EXAMPLE 7

The guidewire of any of Examples 1-6, wherein the rotation limiting feature is configured to limit the proximal guidewire portion from rotating more than two full revolutions relative to the distal guidewire portion.

### EXAMPLE 8

The guidewire of any of Examples 1-7, wherein the rotation limiting feature is configured to limit the proximal guidewire portion from rotating more than three full revolutions relative to the distal guidewire portion.

### EXAMPLE 9

The guidewire of any of Examples 1-8, wherein the rotation limiting feature is configured to limit the proximal guidewire portion from rotating more than four full revolutions relative to the distal guidewire portion.

### EXAMPLE 10

The guidewire of any of Examples 1-9, wherein the proximal illumination fiber defines a first fiber diameter and the at least one distal illumination fiber defines a second fiber diameter, wherein the first fiber diameter is larger than the second fiber diameter.

### EXAMPLE 11

The guidewire of Example 10, wherein the proximal illumination fiber and the at least one distal illumination fiber are arranged in a concentric orientation.

### EXAMPLE 12

The guidewire of any of Examples 1-11, wherein the at least one distal illumination fiber defines a first fiber diameter and the proximal illumination fiber defines a second fiber diameter, wherein the first fiber diameter is larger than the second fiber diameter.

### EXAMPLE 13

The guidewire of Example 12, wherein the proximal illumination fiber and the at least one distal illumination fiber are arranged in a coaxial orientation.

### EXAMPLE 14

The guidewire of any of Examples 1-13, wherein at least one of the proximal illumination fiber or the at least one distal illumination fiber is defined by two or more optical fibers.

### EXAMPLE 15

The guidewire of any of Examples 1-14, wherein the distal guidewire portion includes a coil, wherein an inner core of the coil defines the lumen.

### EXAMPLE 16

A guidewire, comprising: (a) a proximal guidewire portion, including: (i) a first lumen, (ii) a proximal illumination fiber disposed within the first lumen, wherein the proximal illumination fiber defines a first diameter, and (iii) a first connection portion at a distal end of the proximal guidewire portion; and (b) a distal guidewire portion configured to coaxially align with the proximal guidewire portion to define a longitudinal axis, including: (i) a second lumen, (ii) a distal illumination fiber disposed within the second lumen, wherein the distal illumination fiber defines a second diameter, and (iii) a second connection portion at a proximal end of the distal guidewire portion; wherein the first connection portion is configured to couple to the second connection portion to define a rotatable slip coupling; wherein the distal guidewire portion is configured to rotate about the longitudinal axis relative to the proximal guidewire portion; and wherein the first diameter of the proximal illumination fiber is larger than the second diameter of the distal illumination fiber.

### EXAMPLE 17

The guidewire of Example 16, wherein the slip coupling includes a rotation limiting feature configured to limit the rotation of the distal guidewire portion about the longitudinal axis relative to the proximal guidewire portion.

### EXAMPLE 18

The guidewire of Example 17, further comprising: (a) a first threading defined by the first connection portion; and (b) a second threading defined by the second connection portion and configured to form a threaded connection with the first threading.

### EXAMPLE 19

The guidewire of Example 18, wherein the rotation limiting feature includes the threaded connection.

### EXAMPLE 20

The guidewire of any of Examples 16-19, further comprising an illumination source coupled to a proximal end of the proximal illumination fiber.

### EXAMPLE 21

The guidewire of Example 20, wherein a distal end of the distal guidewire portion includes an atraumatic lens configured to emanate light from the illumination source.

### EXAMPLE 22

The guidewire of any of Examples 16-21, wherein the slip coupling is configured to provide electrical continuity between the proximal guidewire portion and the distal guidewire portion.

### EXAMPLE 23

A guidewire, comprising: (a) a proximal guidewire portion, including: (i) a first lumen, and (ii) a proximal illumination fiber disposed within the first lumen; (b) a distal guidewire portion configured to coaxially align with the proximal guidewire portion to define a longitudinal axis, including: (i) a second lumen, and (ii) a distal illumination fiber disposed within the second lumen; and (c) a slip coupling configured to couple a distal end of the proximal guidewire portion to a proximal end of the distal guidewire portion, wherein the distal guidewire portion is rotatable about the longitudinal axis relative to the proximal guidewire portion, wherein the slip coupling includes a rotation limiting feature configured to limit the rotation of the distal guidewire portion about the longitudinal axis to a predefined amount of revolutions in a single rotational direction.

### EXAMPLE 24

The guidewire of Example 23, further comprising an illumination source coupled to a proximal end of the proximal illumination fiber.

### EXAMPLE 25

The guidewire of Example 24, wherein a distal end of the distal guidewire portion includes an atraumatic lens configured to emanate light from the illumination source.

### EXAMPLE 26

The guidewire of any of Examples 23-25, wherein the slip coupling is configured to provide electrical continuity between the proximal guidewire portion and the distal guidewire portion.

### EXAMPLE 27

The guidewire of any of Examples 23-26, further comprising: (a) a first threading defined by a distal end of the proximal guidewire portion; and (b) a second threading defined by a proximal end of the distal guidewire portion and configured to form a threaded connection with the first threading.

### EXAMPLE 28

The guidewire of Example 27, wherein the rotation limiting feature includes the threaded connection.

### EXAMPLE 29

The guidewire of any of Examples 23-28, wherein the rotation limiting feature is configured to limit the proximal guidewire portion from rotating more than two full revolutions in the single rotational direction relative to the distal guidewire portion.

### EXAMPLE 30

The guidewire of any of Examples 23-29, wherein the rotation limiting feature is configured to limit the proximal guidewire portion from rotating more than three full revolutions in the single rotational direction relative to the distal guidewire portion.

### EXAMPLE 31

The guidewire of any of Examples 23-30, wherein the rotation limiting feature is configured to limit the proximal guidewire portion from rotating more than four full revolutions in the single rotational direction relative to the distal guidewire portion.

### EXAMPLE 32

The guidewire of any of Examples 23-31, wherein the proximal illumination fiber defines a first fiber diameter and the distal illumination fiber defines a second fiber diameter, wherein the first fiber diameter is larger than the second fiber diameter.

### EXAMPLE 33

The guidewire of Example 32, wherein the proximal illumination fiber and the distal illumination fiber are arranged in a coaxial orientation.

### EXAMPLE 34

The guidewire of any of Examples 23-33, wherein the distal illumination fiber defines a first fiber diameter and the proximal illumination fiber defines a second fiber diameter, wherein the first fiber diameter is larger than the second fiber diameter.

### EXAMPLE 35

The guidewire of Example 34, wherein the proximal illumination fiber and the distal illumination fiber are arranged in a coaxial orientation.

### EXAMPLE 36

The guidewire of any of Examples 23-35, at least one of the proximal illumination fiber or the distal illumination fiber includes a cable comprising two or more optical fibers.

### EXAMPLE 37

The guidewire of any of Examples 23-36, wherein the distal guidewire portion includes a coil, wherein an inner core of the coil defines the lumen.

### EXAMPLE 38

A guidewire, comprising: (a) a proximal guidewire portion, including: (i) a first optical fiber, and (ii) a first connection portion at a distal end of the proximal guidewire portion; and (b) a distal guidewire portion configured to coaxially align with the proximal guidewire portion to define a longitudinal axis, including: (i) at least two optical fibers, and (ii) a second connection portion at a proximal end of the distal guidewire portion; wherein the first connection portion is configured to couple to the second connection portion to define a rotatable slip coupling; wherein the distal guidewire portion is configured to rotate about the longitudinal axis relative to the proximal guidewire portion at the slip coupling; and wherein the slip coupling is configured to provide optical continuity from the first optical fiber of the proximal guidewire portion to the at least two optical fibers of the distal guidewire portion.

### EXAMPLE 39

The guidewire of Example 39, wherein the slip coupling includes a rotation limiting feature configured to limit the rotation of the distal guidewire portion about the longitudinal axis relative to the proximal guidewire portion.

### EXAMPLE 40

The guidewire of any one or more of Examples 38 through 39, the at least two optical fibers of the distal guidewire portion including a second optical fiber and a third optical fiber, the second and third optical fibers being laterally offset from each other.

### EXAMPLE 41

The guidewire of any one or more of Examples 38 through 40, the at least two optical fibers of the distal guidewire portion including a second optical fiber and a third optical fiber, the second and third optical fibers being parallel with each other.

### EXAMPLE 42

The guidewire of any one or more of Examples 38 through 41, the proximal guidewire portion further including a core wire, the core wire being configured to prevent stretching of the proximal guidewire portion.

### EXAMPLE 43

The guidewire of any one or more of Examples 38 through 42, the distal guidewire portion further including a core wire, the core wire being configured to prevent stretching of the distal guidewire portion.

### EXAMPLE 44

The guidewire of any one or more of Examples 38 through 43, further comprising a handle assembly, the handle assembly comprising: (i) a body, and (ii) a guidewire actuation assembly, the guidewire actuation assembly is operable to rotate the distal guidewire portion relative to the body while the proximal guidewire portion remains stationary relative to the body.

### EXAMPLE 45

The guidewire of Example 44, the guidewire actuation assembly being further operable to translate the distal and proximal guidewire portions relative to the body.

### EXAMPLE 46

The guidewire of any one or more of Examples 44 through 45, further comprising a guide shaft extending distally relative to the body, the distal guidewire portion being disposed in the guide shaft.

### EXAMPLE 47

The guidewire of Example 46, further comprising a dilation catheter slidably disposed relative to the guide shaft, the distal guidewire portion being further disposed in the dilation catheter.

### EXAMPLE 48

The guidewire of Example 47, the dilation catheter including a dilator, the dilator being operable to transition between a non-expanded state and an expanded state, the dilator being configured to fit in a passageway associated with drainage of a paranasal sinus when the dilator is in the non-expanded state, the dilator being configured to dilate a passageway associated with drainage of a paranasal sinus when the dilator is in the expanded state.

### EXAMPLE 49

The guidewire of any one or more of Examples 38 through 48, the distal guidewire portion being configured to fit in a paranasal sinus ostium.

### EXAMPLE 50

The guidewire of any one or more of Examples 38 through 49, the distal guidewire portion further comprising a lens in optical communication with the one or more optical fibers of the distal guidewire portion

IV. Miscellaneous

It should be understood that any of the examples described herein may include various other features in addition to or in lieu of those described above. By way of example only, any of the examples described herein may also include one or more of the various features disclosed in any of the various references that are incorporated by reference herein.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices disclosed herein can be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, versions of the device may be disassembled, and any number of the particular pieces or parts of the device may be selectively replaced or removed in any combination. Upon cleaning and/or replacement of particular parts, versions of the device may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be processed before surgery. First, a new or used instrument may be obtained and if necessary cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a surgical facility. A device may also be sterilized using any other technique known in the art, including but not limited to beta or gamma radiation, ethylene oxide, or steam.

Having shown and described various versions of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1. A guidewire, comprising:
(a) a proximal guidewire portion, including:
(i) a first lumen,
(ii) a proximal illumination fiber disposed within the first lumen, and
(iii) a first connection portion at a distal end of the proximal guidewire portion; and
(b) a distal guidewire portion configured to coaxially align with the proximal guidewire portion to define a longitudinal axis, including:
(i) a second lumen,
(ii) at least one distal illumination fiber disposed within the second lumen, and
(iii) a second connection portion at a proximal end of the distal guidewire portion;
wherein the first connection portion is configured to couple to the second connection portion to define a rotatable slip coupling;
wherein the distal guidewire portion is configured to rotate about the longitudinal axis relative to the proximal guidewire portion at the slip coupling;
wherein the slip coupling is configured to provide optical continuity between the proximal illumination fiber and the at least one distal illumination fiber; and
wherein the slip coupling includes a rotation limiting feature configured to limit the rotation of the distal guidewire portion about the longitudinal axis relative to the proximal guidewire portion.

2. The guidewire of claim 1, further comprising an illumination source coupled to a proximal end of the proximal illumination fiber.

3. The guidewire of claim 2, wherein a distal end of the distal guidewire portion includes an atraumatic lens configured to emanate light from the illumination source.

4. The guidewire of claim 1, wherein the slip coupling is configured to provide electrical continuity between the proximal guidewire portion and the guidewire portion.

5. The guidewire of claim 1, further comprising:
(a) a first threading defined by the first connection portion; and
(b) a second threading defined by the second connection portion and configured to form a threaded connection with the first threading.

6. The guidewire of claim 5, wherein the rotation limiting feature includes the threaded connection.

7. The guidewire of claim 1, wherein the rotation limiting feature is configured to limit the proximal guidewire portion from rotating more than two full revolutions relative to the distal guidewire portion.

8. The guidewire of claim 1, wherein the rotation limiting feature is configured to limit the proximal guidewire portion from rotating more than three full revolutions relative to the distal guidewire portion.

9. The guidewire of claim 1, wherein the rotation limiting feature is configured to limit the proximal guidewire portion from rotating more than four full revolutions relative to the distal guidewire portion.

10. The guidewire of claim 1, wherein the proximal illumination fiber defines a first fiber diameter and the at least one distal illumination fiber defines a second fiber diameter, wherein the first fiber diameter is larger than the second fiber diameter.

11. The guidewire of claim 10, wherein the proximal illumination fiber and the at least one distal illumination fiber are arranged in a concentric orientation.

12. The guidewire of claim 1, wherein the at least one distal illumination fiber defines a first fiber diameter and the proximal illumination fiber defines a second fiber diameter, wherein the first fiber diameter is larger than the second fiber diameter.

13. The guidewire of claim 12, wherein the proximal illumination fiber and the at least one distal illumination fiber are arranged in a coaxial orientation.

14. The guidewire of claim 1, wherein at least one of the proximal illumination fiber or the at least one distal illumination fiber is defined by two or more optical fibers.

15. The guidewire of claim 1, wherein the distal guidewire portion includes a coil, wherein an inner core of the coil defines the lumen.

16. A guidewire, comprising:
(a) a proximal guidewire portion, including:
(i) a first lumen,
(ii) a proximal illumination fiber disposed within the first lumen, wherein the proximal illumination fiber defines a first diameter, and
(iii) a first connection portion at a distal end of the proximal guidewire portion; and
(b) a distal guidewire portion configured to coaxially align with the proximal guidewire portion to define a longitudinal axis, including:
(i) a second lumen,
(ii) a distal illumination fiber disposed within the second lumen, wherein the distal illumination fiber defines a second diameter, and
(iii) a second connection portion at a proximal end of the distal guidewire portion;
wherein the first connection portion is configured to couple to the second connection portion to define a rotatable slip coupling;
wherein the distal guidewire portion is configured to rotate about the longitudinal axis relative to the proximal guidewire portion; and
wherein the first diameter of the proximal illumination fiber is larger than the second diameter of the distal illumination fiber.

17. The guidewire of claim 16, wherein the slip coupling includes a rotation limiting feature configured to limit the rotation of the distal guidewire portion about the longitudinal axis relative to the proximal guidewire portion.

18. The guidewire of claim 16, further comprising:
(a) a first threading defined by the first connection portion; and
(b) a second threading defined by the second connection portion and configured to form a threaded connection with the first threading.

19. The guidewire of claim 16, wherein the slip coupling is configured to provide electrical continuity between the proximal guidewire portion and the distal guidewire portion.

20. A guidewire, comprising:
(a) a proximal guidewire portion, including:
(i) a first optical fiber, and
(ii) a first connection portion at a distal end of the proximal guidewire portion; and
(b) a distal guidewire portion configured to coaxially align with the proximal guidewire portion to define a longitudinal axis, including:
(i) at least two optical fibers, and
(ii) a second connection portion at a proximal end of the distal guidewire portion;
wherein the first connection portion is configured to couple to the second connection portion to define a rotatable slip coupling;
wherein the distal guidewire portion is configured to rotate about the longitudinal axis relative to the proximal guidewire portion at the slip coupling; and
wherein the slip coupling is configured to provide optical continuity from the first optical fiber of the proximal guidewire portion to the at least two optical fibers of the distal guidewire portion.
